(19) United States
(12) Patent Application Publication
(10) Pub. No.: US 2008/0228006 A1
(43) Pub. Date: Sep. 18, 2008

(54) Method For the Reduction of Chlorine-Containing Components in Organic Isocyanates

(75) Inventors: Andreas Job, Koln (DE); Klaus Jost, Dormagen (DE); Hans-Ulrich Dummersdorf, Burscheid (DE)

Correspondence Address: LANXESS CORPORATION, 111 RIDC PARK WEST DRIVE, PITTSBURGH, PA, 15275-1112, US

(73) Assignee: LANXESS DEUTSCHLAND GMBH, Leverkusen (DE)

(21) Appl. No.: 11/792,806

(22) PCT Filed: Dec. 10, 2005
(86) PCT No.: PCT/EP05/13277

§ 371 Date: Jun. 11, 2007

(30) Foreign Application Priority Data
Dec. 22, 2004 (DE) 10 2004 063 221.9
Dec. 22, 2004 (DE) 10 2004 063 222.7

(51) Int. Cl. C07C 263/20

(52) U.S. Cl. 564/264

(57) ABSTRACT

The invention relates to a method for largely eliminating chlorine compounds from organic isocyanates or isocyanate mixtures by contacting said isocyanates or isocyanate mixtures with a water-containing inert gas flow or an organic material having a cation-exchanging effect. The inventive method allows the isocyanates to be gently freed from chorine-containing compounds and is particularly suitable for temperature-sensitive isocyanates.

The present invention relates to a method of largely freeing organic isocyanates or isocyanate mixtures from chlorine compounds by contacting the isocyanates or isocyanate mixtures with a hydrous inert-gas stream or organic materials which possess a cation-exchange effect. The method enables the isocyanates to be freed gently from chlorine-containing compounds and is particularly suitable for temperature-sensitive isocyanates.

Organic isocyanates such as 2,4- and 2,6-diisocyanato-toluene, for example, but also aliphatic isocyanates such as n-butyl isocyanate, for example, frequently contain, as a result of their preparation, impurities, such as, in particular, compounds that contain hydrolyzable chlorine. When the organic isocyanates are put to use, these impurities often give rise to sharp fluctuations in reactivity, which are detrimental to a reproducible industrial use, and a reduced storage stability. The very substantial freeing of the organic isocyanates from the stated impurities is therefore not only of considerable technical importance but also economic importance.

It is known from U.S. Pat. No. 3,912,600, for example, to reduce the level of hydrolyzable chlorine compounds by heating isocyanates, in particular with simultaneous inert-gas stripping and suction removal of the volatile compounds.

Furthermore it is known from EP 482 490, for example, to purify organic isocyanates by means of special distillation and crystallization techniques.

Disadvantages of the processes described are that they can be applied only to thermally insensitive isocyanates and that impurities which are likewise thermally insensitive withstand these operations unscathed.

As well as the purely thermal treatment of isocyanate compounds there have also been descriptions of treatments with adjuvants which allow improved removal of disruptive chlorine compounds. For instance, patents U.S. Pat. No. 3,373,182, U.S. Pat. No. 3,759,971 and U.S. Pat. No. 4,094,894, U.S. Pat. No. 3,458,558 and U.S. Pat. No. 3,264,336 describe adjuvants based on alkali metals or non-alkali metals, such as metal oxides, metal cyanamides, metal hydrides, metal fatty acid esters in the presence of sterically hindered phenols, metal naphthenates, metal silicates, alkali metal carbonates, organometallic compounds, or synthetic zeolites containing (alkali) metal, for example. Generally speaking, however, these adjuvants are difficult to separate from the purified isocyanate, and lead to unwanted metal/metal-ion contamination of the organic isocyanates, which can give rise to a disadvantageous effect in further processing. Moreover, virtually all metals and metal complexes have the effect of unwanted formation of dimers, trimers, and carbodiimides.

The same applies to the use of epoxy compounds (see, e.g., EP 374 932 A and U.S. Pat. No. 6,245,935), formic acid or acetic acid or their derivatives (see, e.g., U.S. Pat. No. 3,799,963) or trimethylsilyl compounds (see, e.g., EP 524 507 A) as adjuvants.

Furthermore, DE-A 12 40 849 discloses a method in which organic isocyanates are purified by the addition of an appropriate amount of water directly to the isocyanate and by subsequent stirring at an elevated temperature, according to example at 150 to 250° C. For safety reasons this procedure is suitable only for small quantities of isocyanate. With larger quantities there is a risk of local overheating at the point of water entry, which at its most serious can lead to explosion of the isocyanate vessel as a result of evolution of carbon dioxide. Moreover, owing to the high temperature, this method is suitable only for thermally insensitive isocyanates.

Because of the above-outlined disadvantages of the methods known in the prior art, therefore, an object which existed was that of providing a broadly applicable method allowing the substantial freeing of the hydrolyzable chlorine fraction in organic isocyanates.

A method has now been found of lowering the level of hydrolyzable chlorine in organic isocyanates or mixtures of organic isocyanates, which is characterized in that liquid organic isocyanates or liquid mixtures of organic isocyanates or solutions of organic isocyanates or solutions of mixtures of organic isocyanates are contacted in a first step with a hydrous inert-gas stream or with organic material with a cation-exchange effect and the resulting reaction mixture in a second step is freed from any solids present.

The scope of the invention encompasses all of the definitions and parameters given above and set out below, whether they be general definitions and parameters or those specified in ranges of preference, in any desired combinations.

The term “hydrolyzable chlorine” as used below is utilized for compounds which react with water to form hydrogen chloride or chloride ions. By way of example, but not exclusively, these are chloroformic amides of the kind which occur as intermediates, for example, in the preparation of isocyanates by phosgenation of amines.

For the method of the invention it is possible to employ any desired organic isocyanates or mixtures of isocyanates, the term “isocyanate” being intended to encompass compounds which contain one, two or even more isocyanate groups.

Preferred organic isocyanates are, for example, mono-isocyanates having aliphatically, cycloaliphatically, araliphatically or aromatically attached isocyanate groups, such as propyl isocyanate, butyl isocyanate, stearyl isocyanate, cyclohexyl isocyanate, benzyl isocyanate, 2-phenylethyl isocyanate, phenyl isocyanate;

diisocyanates having a molecular weight of 140 g/mol to 400 g/mol and having aliphatically, cycloaliphatically, araliphatically and/or aromatically attached isocyanate groups, such as 1,4-diisocyanatobutane, 1,6-diisocyanatohexane (HDI), 2-methyl-1,5-diisocyanatopentane, 1,5-diisocyanato-2,2-dimethylpentane, 2,2,4- and/or 2,4,4-trimethyl-1,6-diisocyanatohexane, 1,10-diisocyanatodecane, 1,3- and 1,4-diisocyanatocyclohexane, 1,3- and 1,4-bis-(isocyanatomethyl)cyclohexane, 1-isocyanato-3,3,5-trimeth-yl-5-isocyanatomethylcyclohexane(isophorone diisocyanate, IPDI), 4,4′-diisocyanatodicyclohexylmethane, 1-isocyanato-1-methyl-4-(3)-isocyanatomethylcyclohexane (IMCI), bis-(isocyanatomethyl)norbornane, 2-methylpentane 2,4-diisocyanate, 1,3- and 1,4-bis(2-isocyanatoprop-2-yl)benzene (TMXDI), 2,4- and 2,6-diisocyanatotoluene (TDI), 2,4′- and 4,4′-diisocyanatodiphenylmethane, 1,5-diisocyanatonaphthalene, dipropylene glycol diisocyanate, 2,4- or 2,6-diisocyanato-1-methylcyclohexane;

triisocyanates and/or higher polyfunctional isocyanates, such as 4-isocyanatomethyloctane1,8-diisocyanate(nonane triisocyanate), 1,6,11-undecane triisocyanate, 3-iso-cyanatomethylhexamethylene 1,6-diisocyanate

and also any desired mixtures of the above organic isocyanates.

Particular preference is given to those of the abovementioned organic isocyanates having a molecular weight of 85 to 400 g/mol, very particular preference to those having a molecular weight of 85 to 279 g/mol.

Still further preferred are isopropyl isocyanate and n-butyl isocyanate.

If liquid organic isocyanates or liquid mixtures of organic isocyanates or solutions of organic isocyanates or solutions of mixtures of organic isocyanates with organic material having a cation-exchange effect are contacted in step 1 of the method of the invention, the following applies:

Preference is given to using liquid organic isocyanates or liquid mixtures of organic isocyanates, particular preference to using liquid organic isocyanates. The liquid aggregate state here refers to the state at the chosen reaction temperature.

The organic material having a cation-exchange effect may, for example, be any polymeric, organic material which based on an aqueous comparison scale contains acidic groups.

The organic material used ought additionally to be largely free of water and other isocyanate-reactive solvents and admixtures, and ought to be separable from the isocyanates by common separation methods such as filtration, sedimentation or centrifugation, for example, and ought to be largely free from heavy metal ions.

Preference is given to using gel-like or macroporous, organic materials having a cation-exchange effect. Such materials are known for example from DE-A 1113570.

The following may be mentioned by way of representation of suitable materials: Lewatite® SC 102, SC 104, SC 108, SPC 118 (all from Bayer AG) or cation-exchange Amberlite® or Duolite® (both from Rohm and Haas).

Preferred materials for the purposes of the method of the invention are the above-described organic cation-exchange materials whose matrix has been obtained by polymerization (copolymers of styrene and divinylbenzene and also of (meth)acrylates and divinylbenzene).

Particular preference is given in the method of the invention to organic, cation-exchange materials whose matrix has been obtained by polymerization (copolymers of styrene and divinylbenzene) and which contain acidic groups, sulfonic acid groups for example.

Substantially free from water means, for the purposes of the method of the invention, that the fraction by Karl-Fischer titration is <5%, preferably <3% and with particular preference <1% by weight. This drying that may be necessary may take place in conventional manner by means for example of replacement of the water by an inert solvent which is not reactive with isocyanates, or by drying of the materials, preferably under reduced pressure.

Substantially free from isocyanate-reactive solvents and admixtures is intended to mean, for the purposes of the method of the invention, a level of such compounds that is below 5%, preferably below 3% and with particular preference below 1% by weight.

Substantially free from heavy metal ions is intended to mean, in the context of the method of the invention, a level of such ions an amount of less than 0.5%, preferably less than 0.3%, and with particular preference less than 0.1% by weight.

The materials having a cation-exchange effect that are used in the method of the invention can be used either in the form of solids without solvents or in the form of a suspension in an inert, non-isocyanate-reactive solvent.

The materials having a cation-exchange effect that are used in the method of the invention can be regenerated by means of suitable method steps and used again in the method of the invention.

The materials having a cation-exchange effect are used preferably in an amount of more-than 0.1%, with particular preference in an amount of 0.5% to 50%, and with particular preference in an amount of 1.0% to 10%, based on the weight of the isocyanate to be purified.

The reaction temperature is for example −10 to 200° C., preferably 20° C. to 140° C., with particular preference 40 to 100° C., and with still further preference 50 to 80° C.

The contacting of the liquid organic isocyanates or liquid mixtures of organic isocyanates or solutions of organic isocynates or solutions of mixtures of organic isocyanates with the organic materials having a cation-exchange effect takes place for example, depending on the batch size, for a duration of 5 minutes to 48 hours.

In accordance with the invention the contacting of the isocyanate compounds with the cation-exchange materials described can take place in such a way that the cation-exchange organic material serves as the stationary phase in a chromatography column and the corresponding isocyanate compound flows through under defined conditions.

With a filter unit integrated in this column technique, the method can also be operated in continuous mode in such or similar apparatus.

If in step 1 of the method of the invention liquid organic isocyanates or liquid mixtures of organic isocyanates or solutions of organic isocyanates or solutions of mixtures of organic isocyanates are contacted with a hydrous inert-gas stream, the following applies:

Gases which can be used as the inert gas for the method of the invention are all gases which under typical reaction conditions do not react with water, hydrogen chloride, amines or isocyanates, such as, for example, nitrogen, noble gases, such as argon, for example, carbon dioxide or mixtures of such gases.

Solvents which can be used are, if necessary, all organic solvents which under typical reaction conditions do not react with water, hydrogen chloride, amines or isocyanates.

Preference is given to using liquid organic isocyanates or liquid mixtures of organic isocyanates, particular preference to using liquid organic isocyanates. The liquid aggregate state here refers to the state at the chosen reaction temperature.

The reaction temperature is for example −10 to 200° C., preferably 0° C. to 120° C., with particular preference 10 to 80° C.

The contacting of the liquid organic isocyanates or liquid mixtures of organic isocyanates or solutions of organic isocyanates or solutions of mixtures of organic isocyanates with a hydrous inert-gas stream may take place for example by passing the hydrous inert-gas stream into or over the isocyanates.

Hydrous means, that the inert-gas stream has a certain partial pressure of water vapor and/or that a mist is used; preferably the hydrous inert-gas stream has a certain partial pressure of water vapor.

The amount of water contacted with the isocyanates via the inert-gas stream is/can be adjusted in conventional manner advantageously by way of the water saturation curve of the inert gas at different temperatures, and is for example 0.5 to 2 mol, preferably 0.8 to 1.4 mol and with particular preference 0.9 to 1.2 mol, per mole of hydrolyzable chlorine contained in the isocyanate.

The contacting of the liquid organic iso cyanates or liquid mixtures of organic isocyanates or solutions of organic isocyanates or solutions of mixtures of organic isocyanates with a hydrous inert-gas stream takes place, as a function of the batch size, for a duration of 5 minutes to 48 hours, for example.

For both method variants in step one the following applies:

Before, during or after step 1 it is also possible to carry out other purification processes, in order for example to remove coloring components and byproducts. Such processes include treatments and/or lightening, using reducing or oxidizing agents for example, and also treatment with adsorbents such as activated carbon and/or bleaching earths and/or silicas. Such lightenings may exert a further positive effect on the lowering of the fraction of hydrolyzable chlorine in the isocyanate compound. The isocyanate that is present after the chlorine content has been lowered by the method of the invention may, furthermore, be subjected in one preferred embodiment to a further purifying operation, by distillation. The distillation in this case takes place preferably under a pressure of, for example, 0.001 mbar to 500 mbar, in the form for example of a still distillation or distillation by means of a thin-film evaporator.

Contacting is preferably carried out until the purified isocyanates or isocyanate mixtures have a hydrolyzable chlorine content of less than 180 ppm.

For step two of the method of the invention the following applies for both variants of the first step:

In the second step of the method of the invention the reaction mixture resulting from step 1 is freed from solids present, in particular from—where appropriate—the organic material having a cation-exchange effect.

The removal of solids can be effected conventionally by means for example of sedimentation, centrifugation or filtration, preference being given to filtration, especially pressure filtration.

It can be of advantage to conduct the two steps of the method of the invention in a reactor/filtration unit combination, where appropriate in continuous mode.

The purified isocyanates or isocyanate mixtures obtainable in accordance, with the invention typically have a hydrolyzable chlorine content of less than 400 ppm and a very high purity. They can therefore be used, for example, for preparing particularly pure oligomeric polyisocyanates or prepolymers or as intermediates for polyurethane moldings and coating materials.

The advantage of the method of the invention is in that disruptive chlorine compounds in the organic isocyanates or isocyanate mixtures for purification can be removed very selectively, under conditions which are easily controllable technically and mild, and without substantial formation of byproducts. The method is also suitable for the demanding workup of aliphatic isocyanates.

### EXAMPLES

The working examples below illustrate the invention. They should not be interpreted as a restriction. The reported values of hydrolyzable chlorine (HC values) are based on the weight. The products obtained were analyzed by means of gas chromatography (area % method, instrument: HP-5890 Series II, column: SE-30).

### Example 1

A three-neck flask is charged with 426.8 g of n-butyl isocyanate (HC value: 2056 ppm, purity by GC analysis: 98.7%). Through a sintered glass frit (pore 3) a stream of nitrogen is passed at 10 l per hour through the initial isocyanate charge, after the latter has been heated to 60° C., the stream of nitrogen being passed beforehand at 23° C., for moistening, through a washbottle filled with water. 30 minutes after the start of the experiment, a solid begins to precipitate. After 3.5 hours, a sample is taken from the supernatant solution for determination of HC value (the analysis shows an HC value of 511 ppm). After a total of 11 h the introduction of nitrogen is shut off, the batch is cooled to RT and filtration is carried out. This gives a clear liquid having an HC value of 146 ppm, a purity of 99.2% by GC analysis, and a yield of 94% of theory.

### Example 2

A three-neck flask is charged with 3804 g of n-butyl isocyanate (HC value: 1300 ppm, purity by GC analysis: 99.5%). Through a sintered glass frit (pore 2) a stream of nitrogen is passed at about 9-10 l per hour through the initial isocyanate charge, after the latter has been heated to 60° C., the stream of nitrogen being passed beforehand at 23° C., for moistening, through a washbottle filled with water. After a total of 15.5 hours the introduction of nitrogen is shut off (water consumption in the washbottle: 3.5 g), the batch is cooled to RT and filtration is carried out. This gives a clear liquid having an HC value of 100 ppm, a purity of 99.5% by GC analysis, and a yield of 96% of theory.

### Example 3 (Inventive)

### Examples 4 to 6 (for Comparison)

A gastight bottle is charged with 80 g of n-butyl isocyanate. Then 5.1 g of Lewatit® SC 102 (cation exchange resin) are introduced into the vessel. This mixture is heated at 60° C. After 5 hours a sample is taken from the supernatant solution for HC-value determination of the fraction of hydrolyzable chlorine (HC value). After a total of 15 hours, the Lewatit® is removed by filtration and subsequently the clear liquid is analyzed by gas chromatography. The results are documented in table 1.

| | | Example 4 | | Example 6 |
|---|---|---|---|---|
| | | (for | Example 5 (for | (for |
| Analysis | Example 3 | comparison) | comparison) | comparison |
| HC value (feed | 2478 ppm | 2478 ppm | 2478 ppm | 2478 ppm |
| material) | | | | |
| Purity (GC of | 98.7% | 98.7% | 98.7% | 98.7% |
| reactant) | | | | |
| Adjuvant | Lewatit ® | Silver wool | Copper wire | None |
| | SC 102 | | | |
| Amount added | 5.1 g | 5.0 g | 5.2 g | 0 |
| HC value of | 291 ppm | 2243 ppm | 1820 ppm | 2492 ppm |
| product (after | | | | |
| 5 h) | | | | |
| HC value of | 135 ppm | 1818 ppm | 1195 ppm | 2422 ppm |
| product (after | | | | |
| 15 h) | | | | |
| Color of | Colorless | Colorless | Green-brown | Colorless |
| solution | | | | |
| (after 15 h) | | | | |
| Purity (GC of | 99.0% | n.d. | n.d. | n.d. |
| product) | | | | |

The silver wool was acquired commercially (Aldrich: catalogue no. 295744) The copper wire was taken from an earthing cable.

### Example 7

A gastight bottle is charged with 50 g of n-butyl isocyanate (HC value: 2471 ppm, content by GC: 98.7%) Then 3.0 g of Lewatit® SC 108 (cation exchange resin) are introduced into the vessel. This mixture is heated at 60° C. for 5 h. Subsequently the Lewatit® is removed by filtration and the clear liquid is analyzed for HC value and isocyanate content. An HC value of 316 ppm and a purity by GC analysis of 99.0% are found.

### Example 8 (for Comparison)

A gastight bottle is charged with 50 g of n-butyl isocyanate (HC value: 2471 ppm, content by GC: 98.7%). Then 3.0 g of Lewatit® MP 62 (anion exchange resin) are introduced into the vessel. This mixture is heated at 60° C. for 5 h. Subsequently the Lewatit® is removed by filtration and the clear liquid is analyzed for HC value and isocyanate content. An HC value of 48 ppm and a purity by GC analysis of 92.4% are found. Here there is a distinct deterioration noted in the purity of the isocyanate.

### Example 9

In a heatable glass column, 39 g of Lewatit® SC 102 (cation exchange resin) are fixed between Raschig rings made of glass (height of the Lewatit® layer: 105 mm). 1 l of isocyanate per hour is pumped from a three-neck flask through the column, which is heated at 60° C.; the isocyanate runs from the overflow of the column back into the three-neck flask. At regular intervals, samples are taken from the three-neck flask and are analyzed for the HC value. The times and results of the respective analyses are reported in table 2. After a total of 29 hours the experiment is ended and the isocyanate obtained is analyzed for purity. A content of 99.5% is measured by GC analysis.

TABLE 2

| Samples | HC values |
|---|---|
| Starting value | 1127 ppm |
| 13 hours | 220 ppm |
| 22 hours | 135 ppm |
| 29 hours | 100 ppm |

1. A method of lowering the level of hydrolyzable chlorine in organic isocyanates or mixtures of organic isocyanates, characterized in that liquid organic isocyanates or liquid mixtures of organic isocyanates or solutions of organic isocyanates or solutions of mixtures of organic isocyanates are contacted in a first step with a hydrous inert-gas stream or with organic material with a cation-exchange effect and the resulting reaction mixture in a second step is freed from any solids present.

2. The method of claim 1, characterized in that in order to lower the level of hydrolyzable chlorine in organic isocyanates or mixtures of organic isocyanates, characterized in that liquid organic isocyanates or liquid mixtures of organic isocyanates or solutions of organic isocyanates or solutions of mixtures of organic isocyanates are contacted in a first step with a hydrous inert-gas stream.

3. The method of claim 2, characterized in that organic isocyanates used are monoisocyanates having aliphatically, cycloaliphatically, araliphatically or aromatically attached isocyanate groups diisocyanates having a molecular weight of 140 g/mol to 400 g/mol and having aliphatically, cycloaliphatically, araliphatically and/or aromatically attached isocyanate groups, triisocyanates and/or higher polyfunctional isocyanates, and any desired mixtures of the above organic isocyanates.

4. The method of at least one of claims 1 to 3, characterized in that organic isocyanates are isopropyl isocyanate or n-butyl isocyanate.

5. The method of at least one of claims 1 to 4, characterized in that liquid organic isocyanates or liquid mixtures of organic isocyanates are used, the liquid aggregate state relating to the state at the chosen reaction temperature.

6. The method of at least one of claims 1 to 5, characterized in that the reaction temperature of the first step is −10 to 200° C.

7. The method of at least one of claims 1 to 6, characterized in that before, during or after step 1 other purification methods as well are carried out.

8. The method of at least one of claims 1 to 7, characterized in that both steps are carried out in a reactor/filtration unit combination.

9. The method of at least one of claims 1 to 8, characterized in that it is carried out until the purified isocyanates or isocyanate mixtures have a hydrolyzable chlorine content of less than 180 ppm.

10. The use of isocyanates prepared according to one or more of claims 1 to 9 for preparing oligomeric polyisocyanates, prepolymers intermediates for polyurethanes and coating materials.
